(19) United States
(12) Patent Application Publication
(10) Pub. No.: US 2026/0069449 A1
(43) Pub. Date: Mar. 12, 2026

(54) FLUID COLLECTION SYSTEM INCLUDING A GARMENT AND A FLUID COLLECTION DEVICE

(71) Applicant: PUREWICK CORPORATION, Covington, GA (US)

(72) Inventors: Jeff Edward Franklin, Hamilton, OH (US); Brandt Davis, Milford, OH (US); Michael Lambert, Snellville, GA (US); Kevin Michael Knollman, West Chester, OH (US); Kyra Kormos Wesner, Loveland, OH (US); Michelle Schnell, Hamilton, OH (US); Mackenzie Brooke Weir, Chardon, OH (US); Joseph Andrew Filbrun, Cincinatti, OH (US)

(21) Appl. No.: 19/329,723

(22) Filed: Sep. 16, 2025

Related U.S. Application Data
(63) Continuation of application No. 18/007,105, now Pat. No. 12,440,371.
(60) Provisional application No. 63/061,834, filed on Aug. 6, 2020.

(51) Int. Cl. A61F 5/455; A61F 5/453

(52) U.S. Cl. CPC A61F 5/455; A61F 5/453

(57) ABSTRACT

Example fluid collection systems and methods of use are described. The fluid collection system includes a garment and a fluid collection device. The garment is configured to be worn on a pelvis of a user, and includes at least a crotch portion having an inner surface and at least one of a strap partially defining a through hole between the strap and the inner surface or a pocket on the inner surface of the crotch portion. The at least one of the strap or the pocket are positioned on the crotch portion of the garment and the fluid collection device is sized to position the opening and the fluid permeable body proximate to a urethra of the user.

### CROSS-REFERENCE TO RELATED APPLICATIONS

This application is a continuation of U.S. Application No. Ser. No. 18/007,105 filed on Jan. 27, 2023, which is a U.S. Nationalization of PCT International Application No. PCT/US2021/044699 filed on Aug. 5, 2021, which claims priority to U.S. Provisional Ser. No. 63/061,834 filed on Aug. 6, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters can be uncomfortable, painful, and can cause urinary tract infections.

Thus, users and manufacturers of fluid collection devices continue to seek new and improved devices, systems, and methods to collect urine.

### SUMMARY

Embodiments disclosed herein are fluid collection systems and methods of use. In an embodiment, a fluid collection system includes a garment and fluid collection device. The garment is configured to be worn on a pelvis of a user. The garment includes at least a crotch portion having an inner surface and at least one of a strap partially defining a through hole between the strap and the inner surface or a pocket on the inner surface of the crotch portion. The fluid collection device has at least a portion sized and shaped to fit within one or more of the at least one of the through hold or the pocket. The fluid collection device including a fluid impermeable barrier and a fluid permeable body. The fluid impermeable barrier at least partially defines a chamber, an opening extending longitudinally along the fluid impermeable barrier and configured to be positioned adjacent to a urethra of the user, and an aperture configured to receive a conduit therethrough. The fluid permeable body is positioned at least partially within the chamber to extend across at least a portion of the opening and configured to wick fluid away from the opening. The at least one of the strap or the pocket are positioned on the crotch portion of the garment and the fluid collection device is sized to position the opening and the fluid permeable body at least proximate to the urethra of the user.

In an embodiment, a fluid collection system includes a garment, a fluid collection device, and a conduit. The garment is configured to be worn on a pelvis of a user. The garment includes at least a crotch portion having an inner surface and a strap secured to the inner surface of the garment and at least partially defining a through hole between the strap and the inner surface. The fluid collection device includes a fluid impermeable barrier and a fluid permeable body. The fluid impermeable barrier at least partially defines a chamber, an opening extending longitudinally along the fluid impermeable barrier and configured to be positioned adjacent to a urethra of the user, and an aperture configured to receive a conduit therethrough. The fluid permeable body is positioned at least partially within the chamber to extend across at least a portion of the opening and configured to wick fluid away from the opening. The conduit extends through the aperture such that a first portion of the conduit is disposed within the chamber and a second portion of the conduit is disposed outside the chamber. The conduit has at least a portion sized and shaped to fit within the through hole. The strap is positioned on the crotch portion of the garment and the fluid collection device is sized to position the opening and the fluid permeable body at least proximate to the urethra of the user.

In an embodiment, a method of collecting fluid includes securing a fluid collection device to an inner surface of a garment. The fluid collection device is secured to the inner surface of the garment by at least one of securing a first end region of a fluid impermeable barrier of the fluid collection device in a through hole defined by a portion of the inner surface and a strap secured to the inner surface, or inserting a second end region of the fluid impermeable barrier of the fluid collection device in a pocket on the inner surface of the pocket. The method also includes disposing the garment on the wearer such that an opening in the fluid impermeable barrier and a fluid permeable body disposed in a chamber defined by the fluid impermeable body are positioned adjacent to the urethra of the wearer. The method also includes receiving fluids from the urethra into the chamber.

In an embodiment, a garment configured to be worn on a pelvis of a user to collect fluid includes a waistband and at least a portion having an inner surface. The crotch portion also includes at least one of a strap or a pocket. The strap partially defines a through hole between the strap and the inner surface, the through hole being sized and dimensioned to secure a conduit or a first end region of a fluid impermeable barrier of a fluid collection device therein. The pocket is on the inner surface of the crotch portion and is sized and dimensioned to secure the second end region of a fluid impermeable barrier of the fluid collection device therein. The at least one of the strap or the pocket are positioned on the inner surface of the crotch portion to position an opening and a fluid permeable body of the fluid collection device at least proximate to a urethra of the user when the fluid collection device is secured to the inner surface with the at least one of the strap or the pocket.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a front view of a garment for fluid collection worn on a user, according to an embodiment.

FIG. 2A is an isometric view of a fluid collection device, according to an embodiment.

FIGS. 2B and 2C are cross-sectional views of the female fluid collection device of FIG. 1 taken along line 2-2 thereof, according to various embodiments.

FIG. 2D is an exploded view of the female fluid collection device of FIG. 2A.

FIG. 3A is a front view of the garment for fluid collection of FIG. 1.

FIG. 3B is a cross-sectional side view of the garment of FIG. 3A taken along line 3-3 thereof.

FIG. 3C is a top view of the garment of FIG. 3A.

FIG. 4A is a front view of a garment for fluid collection worn on a user, according to an embodiment.

FIGS. 4B and 4C are views of the garment of FIG. 4A with a fluid collection device, according to an embodiment.

FIGS. 5-7 are front views of garments for fluid collection, according to various embodiments.

FIG. 8 is a block diagram of a system for fluid collection, according to an embodiment.

FIG. 9 is a flow diagram of a method to collect fluid, according to an embodiment.

### DETAILED DESCRIPTION

Embodiments disclosed herein are related to garments carrying fluid collection devices and methods of using the same. The garments may include one or more of breathable materials and/or absorptive materials. The garments include a crotch portion having one or more securement features providing the technical effect of securing a fluid collection device on an inner surface of the crotch portion. The one or more securement features can include various combinations of one or more straps and a pocket configured to allow a user to secure the fluid collection device to the crotch portion of the garment, as well as remove the fluid collection device from the crotch portion. For example, the one or more securement features may include a strap configured to secure a conduit to the inner surface of the crotch portion, a strap configured to secure a proximate or first end region of the fluid impermeable barrier of the fluid collection device to the inner surface of the crotch portion, and/or a pocket configured to secure a distal or second end region of the fluid impermeable barrier to the inner surface of the crotch portion. The securement features and the crotch portion of the garment provide the technical effect of aligning the fluid collection device thereon with one or more anatomical structures (e.g., urethra) of the wearer when the garment is worn.

The fluid collection devices include a fluid impermeable barrier that at least partially defines a chamber therein. The fluid impermeable barrier also defines an opening extending therethrough that is configured to be positioned adjacent to a female urethra or have a male urethra positioned therethrough. In some examples, the garment results in the technical effect of positioning the opening in the fluid impermeable barrier and the fluid permeable body adjacent the urethra and/or next to the skin surround the urethral opening. The fluid collection device includes a conduit for removing fluid from the chamber. The conduit includes an inlet in fluid communication with (e.g., positioned within) the chamber of the fluid collection device and an outlet configured to be in fluid communication with (e.g., fluidly coupled to) a vacuum source or fluid storage container. The fluid collection device also include a tube having a channel extending between an inlet and outlet thereof. The inlet is configured to be in fluid communication with the vacuum source and the outlet is configured to be in fluid communication with a fluid storage (vessel or container), or vice versa. The outlet is positioned downstream from the inlet.

The garments disclosed herein are configured to collect fluids from an individual. The fluids collected by the garments (e.g., via fluid collection devices positioned therein and/or secured thereto) can include urine. The fluids collected by the fluid collection devices can also include at least one of vaginal discharge, penile discharge, reproductive fluids, blood, sweat, or other bodily fluids. The fluid collection garments, systems, and methods disclosed herein can help keep a wearer dry and sanitary while reducing or eliminating the need for more invasive means of collecting fluid emissions such as catheterization.

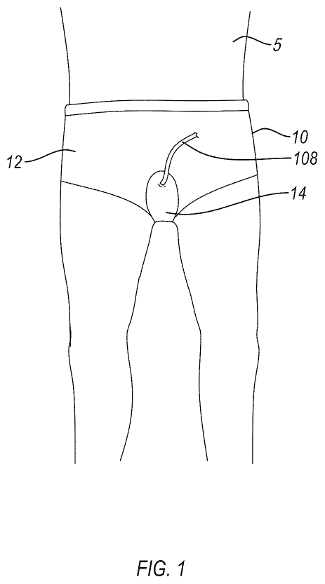

FIG. 1 is a front view of a garment 10 for fluid collection, according to an embodiment. The garment 10 includes a fabric body 12 that is sized and shaped to fit on a pelvis of a wearer or user 5 and maintain position thereon. The garment 10 also includes a crotch portion 14 therein. The crotch portion 14 may be positioned in the crotch of the garment 10 (e.g., a position corresponding to the crotch of the user 5 when the garment 10 is worn). The crotch portion 14 of the garment can include the region of the garment positioned to cover an area extending from the pubic region to the anus or the perineum of the subject when the garment is worn. The crotch portion 14 is positioned over at least the region of the urethra of the user 5 (e.g., within the region of the crotch) when the garment 10 is worn. A fluid collection device (not shown) may be disposed on an inner surface of the crotch portion (described in greater detail below), such that fluids in the region, such as urine, may be collected by the fluid collection device. Accordingly, the garment 10 may provide the technical effect of aligning the fluid collection device with the urethra of the user such that fluids originating therefrom can be collected before soiling clothing or bedding. The garment 10 also includes one or more (e.g., two) leg openings and a waist opening.

The fabric body 12 and/or the crotch portion 14 may include one or more of natural materials such as cotton, wool, silk, rubber, etc. or synthetic materials such as polyamides (e.g., NYLON), poly(ethylene terephthalate), beta-1,4-Glucan-4-Glucanohydrolase (e.g., Rayon), polyester, silicone, a polyether-polyurea copolymer (e.g., SPANDEX), olefin fibers, acrylic fibers, etc. The fabric body 12 and/or the crotch portion 14 may include one or more breathable material and/or one or more absorptive materials. In some examples, at least one of the fabric body 12 and/or the crotch portion 14 include one or more of a mesh fabric, a woven fabric, or a non-woven material. In some embodiments, the fabric body 12 and the crotch portion 14 of the undergarment 10 may include identical material(s). In some embodiments, the fabric body 12 and the crotch portion 14 may include one or more different materials. For example, the undergarment 10 can include one or more portions which include a relatively soft fabric and one or more portions that include a relatively rough fabric. In some examples, the undergarment 10 can include a cotton gusset crotch portion 14 and a nylon body 12. In some examples, the fabric body 12 may include materials configured for user comfort, such as cotton, wool, silk, or synthetic materials such as polyamides (e.g., NYLON), poly(ethylene terephthalate), beta-1,4-Glucan-4-Glucanohydrolase (e.g., Rayon), polyester, silicone, a polyether-polyurea copolymer (e.g., SPANDEX), olefin fibers, acrylic fibers, etc. The crotch portion 14 may include materials configured to absorb fluids or wick fluids away from the skin of the user 5. For example, the crotch portion 14 may consist of or consist essentially of a mesh, a porous, and/or fibrous material, such as hydrophilic polyolefin. Examples of polyolefin that may be used in the crotch portion 14 include, but are not limited to, polyethylene, polypropylene, polyisobutylene, ethylene propylene rubber, ethylene propylene diene monomer, or combinations thereof.

The garment 10 can be shaped as a variety of garments, such as an undergarment, pants, shorts, a jock-strap, adjustable versions of any of the foregoing, or analogues of any of the foregoing. In such examples, stitching, buttons, snaps, or other fasteners can join a plurality of panels which collectively form the garment.

The crotch portion 14 may be positioned on the undergarment 10 such that the crotch portion 14 is located about a urethral region on the pelvis of the user 5 when the garment 10 is worn. For example, the crotch portion 14 can be positioned over or in the crotch of the undergarment 10. The crotch portion 14 can provide the technical effect of securing and retaining a fluid collection device in place over the urethral region on the pelvis of the user 5. The crotch portion 14 may be sown on or in the fabric body 12. The crotch portion 14 may be integrally formed in the fabric body 12 (e.g., weaved into the fabric body 12).

The crotch portion 14 may be releasably and selectively attachable to the fabric body 12. For example, the crotch portion 14 may be affixed to the fabric body by hook and loop fasteners, buttons, snaps, a zipper, magnets, or any other fastener. In such examples, the fabric body 12 can include a male portion of the snap connection, a hook strip, a button, or a magnet disposed in the region around the crotch of the subject, such as around the periphery of the region where a urethra of a wearer may be located while the garment is worn. The piece of fabric defining the crotch portion 14 can include a female portion of the snap connection, a loop strip, a button-hole, or a magnet disposed about a periphery of the piece of fabric. In such examples, the crotch portion 14 can be aligned and retained on the fabric body 12 by the snap connection, hook strip, button, or magnet. Accordingly, the garment 10 can provide a technical effect of a crotch portion 14 that is removable and replaceable, such as to insert, replace, clean, or otherwise service the fluid collection device in the garment 14. Furthermore, the garment 10 can provide a technical effect of a crotch portion 14 that allows removal and replacement of a fluid collection device without removing the garment 10.

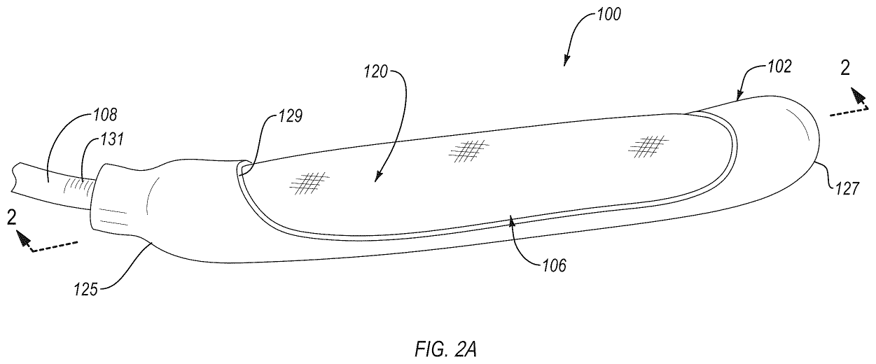

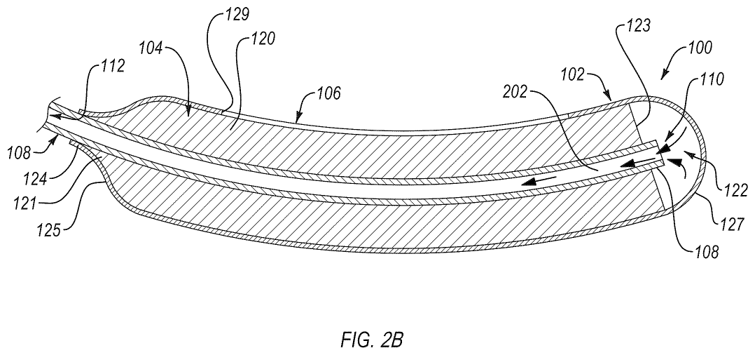

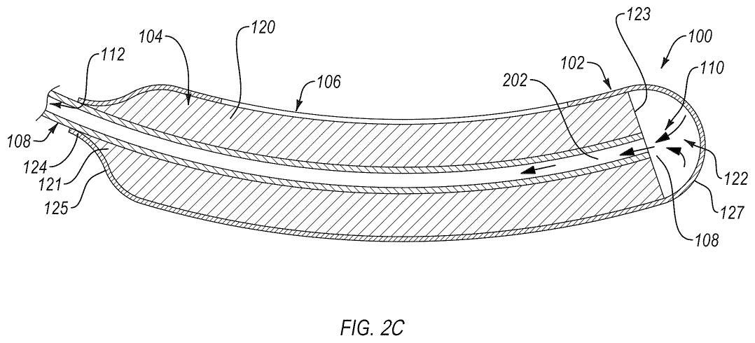

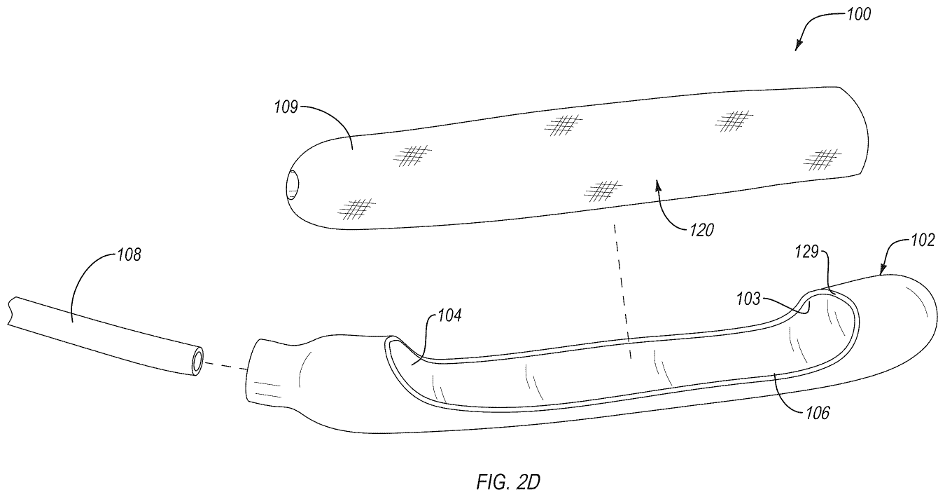

A fluid collection device can be secured the garment 10 (e.g., on the crotch portion 14). For example, the crotch portion 14 may be sized and shaped to accommodate a female fluid collection device or a male fluid collection device, such as any of those disclosed herein. FIG. 2A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 2C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. The opening 106 is formed in and extends longitudinally through the fluid impermeable barrier 102, thereby enabling fluids to enter the chamber 104 from outside of the fluid collection device 100. The opening 106 may be configured to be positioned adjacent to the opening of a female urethra.

The fluid collection device 100 may be positioned at least proximate to the opening of the female urethra and urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., at or near the anus or the vaginal opening) to a second location above the urethral opening (e.g., at or near the clitoris or the pubic hair). The opening 106 may exhibit an elongated shape since the space between the legs of a female is relatively small when the legs of the female are closed, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual. According to an embodiment, a suitable adhesive is a hydrogel layer, such as those disclosed in U.S. Patent Application Publication No. 2017/0189225, the disclosure of which is incorporated herein by reference in its entirety.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” may exclude absorption into the wicking material. The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIGS. 2B and 2C) of void space formed within the chamber 104. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 2D) of the fluid permeable body 120 interfaces with an inner surface 103 (shown in FIG. 2D) of the fluid impermeable barrier 106. A singular fluid permeable body 120 may be advantageous to conventional systems, which typically require an air-laid nonwoven pad covered by a ribbed fabric compression bandage, because a singular fluid permeable body 120 reduced the number of components in the fluid collection device 100, reduces the assembly time of the fluid collection device 100, requires shelf-life data for only a single component, and provides a latex-free single component. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier 102 is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface and a single layer or type of material between the opening 106 and the conduit 108 positioned within the fluid permeable body 120.

In other embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. The permeable properties referred to herein may be wicking, capillary action, diffusion, or other similar properties or processes, and are referred to herein as “permeable” and/or “wicking.” In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating. Such “wicking” may not include absorption into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as about 30 wt% of the dry weight of the wicking material, about 20 wt%, about 10 wt%, about 7 wt%, about 5 wt%, about 3 wt%, about 2 wt%, about 1 wt%, or about 0.5 wt% of the dry weight of the wicking material.

The fluid permeable membrane may also wick the fluid generally towards an interior of the chamber 104, as discussed in more detail below. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous nylon structure (e.g., spun nylon fibers) and a fluid permeable membrane including gauze about or over the porous nylon structure.

In use, the fluid permeable body 120 of the fluid collection device is positioned adjacent to a urethra of the user 150. The fluid permeable body 120 is disposed within a chamber 104 (shown in FIGS. 2B and 2C) of the fluid impermeable barrier 102 of the fluid collection device 100 and is exposed to the urethra of the user 150 through the opening 106 in the fluid collection device 100. The fluid collection device 100 may be secured to the user with any of a number of securing devices. Fluids received in the chamber 104 of the fluid collection device 100 from the urethra may be removed through the conduit 108.

FIG. 2B is a cross-sectional view of the fluid collection device 100 taken along line 2-2 of FIG. 2A. The fluid collection device 100 also includes conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 is positioned in the reservoir 122. However, in other embodiments, the inlet 110 may be positioned flush with or behind an end of the fluid permeable body 120 that partially defines the reservoir 122. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 is an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 2B, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent applicationer. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 2B. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, as shown in FIG. 2C, an end of the conduit 108 may be generally flush or coplanar with the second body end 123 of the fluid permeable body 120. In other embodiments, the end of the conduit 108 may be recessed from the second body end 123 of the fluid permeable body 120. The end of the conduit 108 also may be selectively moveable between partially extending into the reservoir 122 (shown in FIG. 2B) and recessed from or flush with the second body end 123 of the fluid permeable body (shown in FIG. 2C).

When secured to the fluid collection device 100, the conduit 108 is configured to provide fluid communication with and at least partially extend between one or more of a fluid storage containers (not shown) and a portable vacuum source (not shown). For example, the conduit 108 may be configured to be fluidly coupled to and at least partially extend between one or more of the fluid storage containers and the portable vacuum source. In an embodiment, the conduit 108 is configured to be directly connected to the portable vacuum source (not shown). In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container (not shown) or the portable vacuum source (not shown). In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C.R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 2A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e.g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

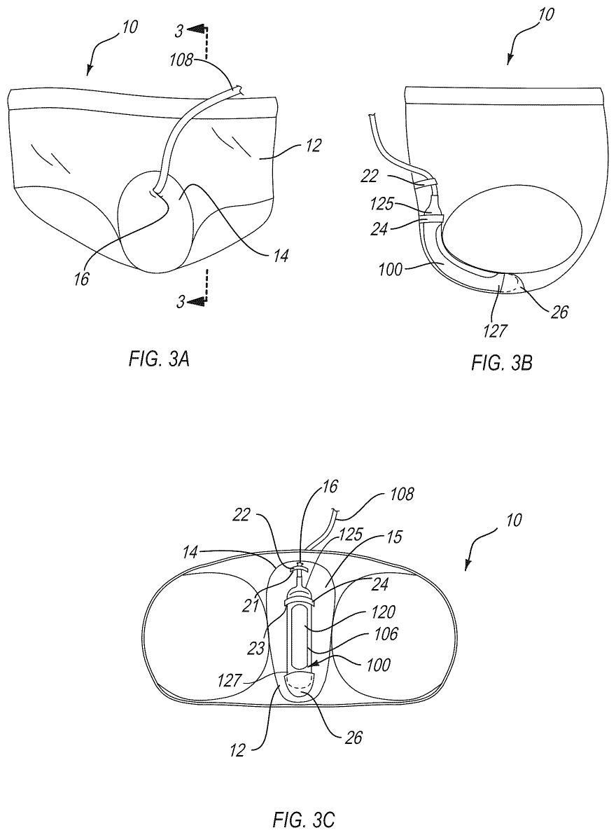

FIGS. 3A-3C are views of the garment 10 of FIG. 1 with a fluid collection device 100, according to an embodiment. Although the fluid collection device 100 described above is shown in FIGS. 3A-3C, the garment 10 may be configured for use other fluid collection devices, including both female and male fluid collection devices. For example, according to an embodiment, a male fluid collection device may be used with the garment 10, such as the male fluid collection devices disclosed in U.S. patent application Ser. No. 16/433,773, the disclosure of which is incorporated herein by reference in its entirety.

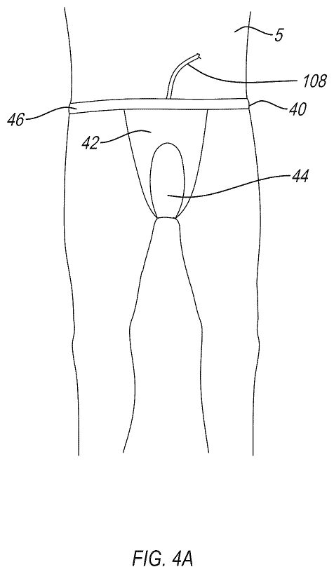

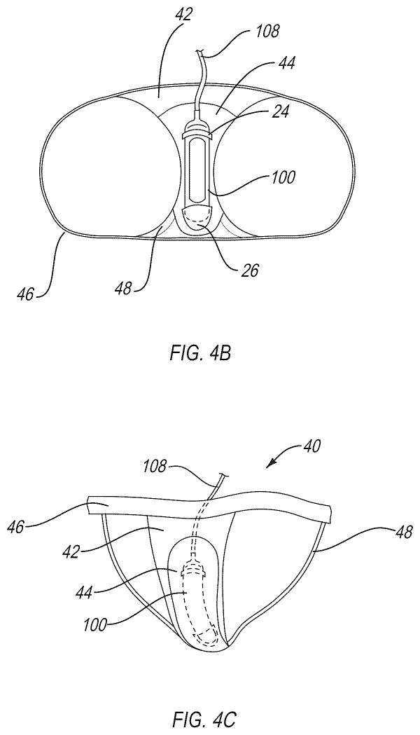

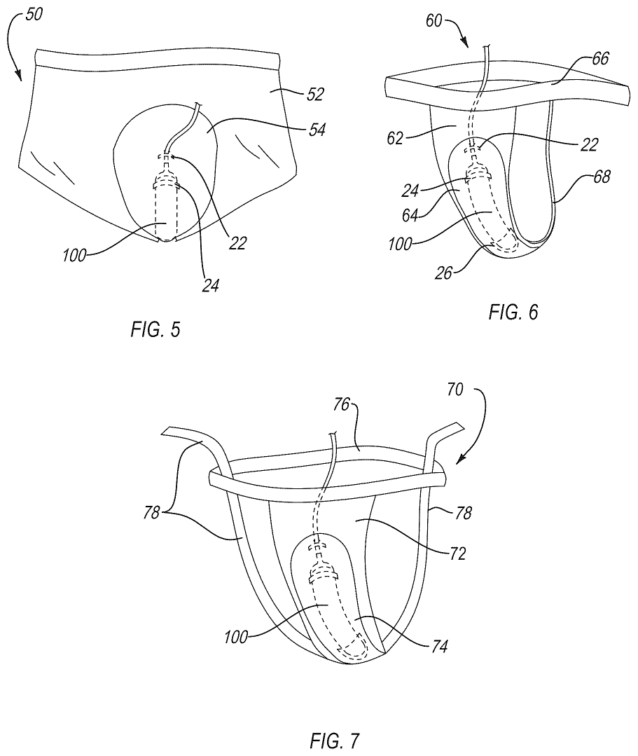

As described above, the garment 10 may include a body, such as a fabric body 12, and a crotch portion 14. The crotch portion 14 may define or otherwise include an opening 16 (shown in FIGS. 3A and 3C) sized and dimensioned to allow the conduit 108 to pass therethrough. The opening 16 may include a diameter generally equal to the diameter of the conduit 108. In some embodiments, the opening 16 may include stretch to allow the conduit 108 to pass therethrough while fitting around the conduit 108. The opening 16, then, may result in a technical effect of allowing insertion and removal of the conduit 108 through the opening 16, as well as retention of the conduit 108 in a preferred position. In some embodiments, the opening 16 is absent (such as FIG. 4A), and the conduit may exit the garment through the waist opening or a leg opening. As shown in FIG. 3C, the crotch portion 14 may fixedly or releasably attach two portions (such as the front and back) of the fabric body 12. In some embodiments, the crotch portion 14 may be entirely surrounded by the fabric body (see, for example, FIG. 5).

With reference to FIGS. 3B and 3C, the garment 10 also includes one or more securement features on the inner surface 15 of the crotch portion 14. For example, the garment 10 includes straps 22, 24 and a pocket 26. While the garment 10 includes all three of the straps 22, 24 and the pocket 26, other embodiments may include any combination of one or more of the straps 22, 24 and the pocket 26. For example, some embodiments of the garment may include only the strap 22, only the strap 24, only the pocket 26, the strap 22 and the pocket 26, or the strap 24 and the pocket 26. Each of the securement features on the inner surface 15 of the crotch portion 14 may include a material that is the same or different from the material of the crotch portion 14. In some embodiments, one or more of the securement features may include an elastic material, silicone, polypropylene, polyethylene, polyethylene terephthalate, a polycarbonate, TPE, rubber, thermoplastic polyurethane, a woven fabric, another suitable material, or combinations thereof. The one or more straps 22, 24 and the pocket 26 result in a technical effect of allowing a user to secure the fluid collection device 100 to the garment 10 in a desired position, remove a soiled fluid collection device 100, and secure a different fluid collection device 100 to the garment 10 in the desired position.

Each of the securement features may be fixedly secured or at least partially detachably secured to the inner surface 15 of the crotch portion 14. For example, one or more of the straps 22, 24 maybe fixedly secured to the inner surface 15 of the crotch portion 14 at two portions of the straps 22, 24, such as the two ends of the straps 22, 24. With the straps 22, 24 fixedly secured to the inner surface 15 of the crotch portion 14 at two portions, through holes 21, 23 are defined or otherwise formed between the straps 22, 24 and the inner surface 15 of the crotch portion 14.

In some embodiments, one or more of the straps 22, 24 may be fixedly secured to the inner surface 15 of the crotch portion 14 at a single portion of the straps 22, 24, such as a single end of the straps 22, 24. For example, the strap 22 may be fixedly secured to the inner surface 15 of the crotch portion 14 at a first end of the strap 22, while a second end of the strap 22 distal to the first end is not fixedly secured to the inner surface 15. The strap 24 may include a similar configuration. When only a single portion or end of the straps 22, 24 is fixedly secured to the inner surface 15 of the crotch portion 14, another portion of the straps 22, 24, such as the other (or second) end of the straps 22, 24 may be configured to detachably or releasably secure to the inner surface 15 of the crotch portion 14. Straps 22, 24 having this configuration may define the through holes 21, 23 before or after the conduit 108 and or the fluid collection device 100 is positioned on the inner surface 15 of the crotch portion 14. Straps 22, 24 having this configuration also may result in the technical effect of securing varying sizes of conduits or fluid collection devices to the inner surface 15 of the crotch portion 14. In some embodiments, one or more of the straps 22, 24 may not be fixedly secured (e.g., fixed securement is absent from one or more of the straps 22, 24), and two portions or ends of the straps 22, 24 are configured to detachably or releaseably secure to the inner surface 15 of the crotch portion 14.

Similar to the straps 22, 24, the pocket 26 maybe fixedly secured to the inner surface 15 of the crotch portion 14 at two portions of the pocket 26, such as two edges of the pocket 26. With the pocket 26 fixedly secured to the inner surface 15 of the crotch portion 14 at two portions, an opening and a cavity is defined or otherwise formed between the pocket 26 and the inner surface 15 of the crotch portion 14. The two edges of the pocket 26 fixedly secured to the inner surface 15 may connect at a distal end of the pocket 26 distal to the opening of the pocket 26. Alternatively, a gap may be positioned between the two edges of the pocket 26 fixedly secured to the inner surface 15.

In some embodiments, the pocket 26 may be fixedly secured to the inner surface 15 of the crotch portion 14 at a single portion of the pocket 26, such as a single edge of the pocket 26. For example, the pocket 26 be fixedly secured to the inner surface 15 of the crotch portion 14 at a first edge of the pocket 26, while a second edge of the pocket distal to the first edge is not fixedly secured to the inner surface 15. When only a single portion or edge of the pocket 26 is fixedly secured to the inner surface 15 of the crotch portion 14, another portion of the pocket 26, such as the other (or second) edge of the pocket may result in the technical effect of detachably or releasably securing the pocket 26 to the inner surface 15 of the crotch portion 14. A pocket 26 having this configuration may define the cavity before or after the fluid collection device 100 is positioned on the inner surface 15 of the crotch portion 14. A pocket 26 having this configuration also may result in the technical effect of securing varying sizes of fluid collection devices to the inner surface 15 of the crotch portion 14. In some embodiments, the pocket 26 may not be fixedly secured (e.g., fixed securement is absent from the pocket), and two portions or edges of the pocket 26 are configured to detachably or releaseably secure to the inner surface 15 of the crotch portion 14.

Securement features that are fixedly secured to the inner surface 15 of the crotch portion 14 may be fixedly secured to the inner surface 15 with one or more of an adhesive, a threaded seam, ultrasonic welding, other suitable securements, or combinations thereof. Securement features that are configured to detachably or releaseably secure to the inner surface 15 of the crotch portion 14 may include one or more of hook and loop fasteners, buttons, snaps, clips, magnets, or any other fastener, with the inner surface 15 of the crotch portion 14 having a corresponding fastener.

The securement features are positioned or are positionable on the inner surface 15 of the crotch portion 14 to provide the technical effect of positioning the opening 106 and the fluid permeable body 120 of the fluid collection device 100 proximate or adjacent to a urethra of the user 5 (e.g., over, on, or covering the urethra of the user 5). Though the user 5 is not shown, FIG. 3B shows how the fluid collection device 100 may bend to conform to the shape in the pelvic region of the user 5 to position the opening 106 and the fluid permeable body 120 of the fluid collection device 100 proximate or adjacent to a urethra of the user 5 when the fluid collection device is secured to inner surface 15 of the crotch portion 14.

In an embodiment, the strap 22 is positioned or positionable on the inner surface 15 of the crotch portion 14 to secure a portion of the conduit 108 to the inner surface 15. The strap 22 at least partially defines the through hole 21 between the strap 22 and the inner surface 14. The conduit 108 includes a portion disposed outside the chamber 104 of the fluid impermeable barrier 102, and the through hole 21 may be sized and dimensioned to hold a segment of that portion of the conduit within the through hole 21.

In an embodiment, the strap 24 is positioned or positionable on the inner surface 15 of the crotch portion 14 to secure the first end region 125 to the inner surface 15. The strap 24 at least partially defines the through hole 23 between the strap 24 and the inner surface 14. The through hole 23 may be sized and dimensioned to hold a portion of the first end region 125 of the fluid impermeable barrier 102 within the through hole 22. The elasticity or flexibility of the strap 24 may result in the strap 24 having the technical effect of stretching around a portion of the first end region 125 to secure the first end region 125 within the through hole 23.

In an embodiment, the pocket 26 is positioned or positionable on the inner surface 15 of the crotch portion 14 to secure at least a portion of the second end region 127 within the pocket 26. The pocket 26 at least partially defines a cavity of the pocket 26. The pocket 26 and the cavity may be sized and dimensioned to hold at least a portion of the second end region 127 of the fluid impermeable barrier 102 within the cavity of the pocket 26. The pocket 26 may extend at least partially from an end of the fluid impermeable barrier 102 to the opening 106 when the second end region 127 is at least partially positioned within the pocket 26. The elasticity or flexibility of the pocket 26 may result in the technical effect of the pocket 26 stretching around a portion of the second end region 127 to secure the second end region 127 within the through cavity of the pocket 26.

While FIGS. 3A-3C show the fluid collection device 100 secured to the garment 10, other fluid collection devices may be secured to the inner surface 15 of the crotch portion 14. For example, straps 22, 24 may secured around the conduit and a portion of a male fluid collection device. Moreover, the garments herein may have different forms including: pants; shorts; undergarments such as briefs, boxer briefs, a thong, a jock strap, etc. ; a sanitary belt; or the like.

Turning now to FIGS. 4A-C, which provide views of a garment 40 for fluid collection, according to an embodiment. The garment 40 may be similar or identical to the garment 10 in one or more aspects, but sized, shaped, and otherwise configured as a jock strap. Unless otherwise noted, the garment 40 may include any of the technical effects described in relation to the garment 10. The garment 40 includes a fabric body 42 that is sized and shaped to fit on a portion of a pelvis of the user 5 and maintain position thereon, as shown in FIG. 4A. The fabric body 42 of the garment 40 may be similar or identical to the fabric body 12 in one or more aspects. The fabric body 42 of the garment 40 includes a crotch portion 44 therein. The crotch portion 44 of the garment 40 may be similar or identical to the crotch portion 14 in one or more aspects. For example, the crotch portion 44 may be positioned in the crotch of the garment 40. The crotch portion 44 is positioned over the region of the urethra of the wearer (e.g., within the region of the crotch) when the garment 40 is worn. The crotch portion 44 includes one or more of the securement features described above in relation to the crotch portion 14. For example, FIGS. 4B and 4C show the crotch portion 44 includes the strap 24 and the pocket 26, and the strap 22 is absent. In other embodiments, the crotch portion 44 may include both of the straps 22, 24 and also the pocket 26. In some embodiments, the crotch portion 44 may include the strap 22 and the pocket 26, and the strap 24 may be absent. The securement features of the crotch portion 44 may include any aspects and technical effects of the securement features of the crotch portion 14, such as fixed securement and/or detachable securement to the inner surface of the crotch portion 44. A fluid collection device (e.g., 100) may be secured to the crotch portion 44, such that fluids in the region (e.g., urine) may be collected by the fluid collection device. The conduit opening may be absent from the crotch portion 44, and the conduit 108 may exit the garment 40 through the opening at the waistband 46 (shown in FIGS. 4A and 4C) or one of the leg openings. When secured to the crotch portion 44, the fluid collection device 100 may take shape around the pelvic region of the user 5, as described above in relation to the garment 10. Accordingly, the garment 40 and securement features of the crotch portion 44 may serve to align the fluid collection device 100 with the urethra of the user such that fluids originating therefrom can be collected before soiling clothing or bedding.

The garment 40 also includes a waistband 46 and two straps 48 extending from the crotch portion 44 to the waistband 48. The straps 48 extend over the buttocks of the user 5, and may be secured directly to crotch portion 44 or may be secured to the fabric body 42. The fabric body 42 may be positioned only at the front of the garment 40 such that the fabric body 42 extends only between the waistband 46 and the crotch portion 44 in the front, and the straps 48 extend between the crotch portion 44 and the waistband 46 in the rear of the garment 40 in the absence of the fabric body 42. In some examples, the fabric body 42 surrounds the crotch portion 44, and the straps 48 may be secured to the fabric body 42.

FIG. 5 is a front view of a garment 50 for fluid collection, according to an embodiment. The garment 50 may be similar or identical to the garment 10 in one or more aspects and, unless otherwise noted, may include any of the technical effects described above in relation to the garment 10. The garment 50 includes a fabric body 52 that is sized and shaped to fit on a pelvis of a wearer and maintain position thereon. The fabric body 52 may be sized, shaped, and configured to form undergarments in the form of boxer briefs or shorts. The fabric body 52 of the garment 50 may be similar or identical to the fabric body 12 in one or more aspects. The garment 50 also includes a crotch portion 54. The crotch portion 54 of the garment 50 may be similar or identical to the crotch portion 14 or the crotch portion 44 in one or more aspects. For example, the crotch portion 54 is positioned over the region of the urethra of the wearer (e.g., within the region of the crotch) when the garment 50 is worn. A fluid collection device (e.g., 100) may be secured to the crotch portion 54 with one or more securement features, such that fluids in the region (e.g., urine) may be collected by the fluid collection device. Accordingly, the garment 50 and crotch portion 54 may serve to align the fluid collection device 100 with the urethra of the user such that fluids originating therefrom can be collected before soiling clothing or bedding.

FIG. 6 is a front view of a garment 60 for fluid collection, according to an embodiment. The garment 60 may be similar or identical to the garment 10, 40, or 50 in one or more aspects and, unless otherwise noted, may include of the technical effects described above in relation to the garment 10. The garment 60 includes a fabric body 62 that is sized and shaped to fit on a pelvis of a wearer and maintain position thereon. The fabric body 62 may be sized, shaped, and configured to form undergarments in the form of thong underwear having a waistband 66 and a back strap 68 that fits between the buttocks of the user 5. The fabric body 62 of the garment 60 may be similar or identical to the fabric body 12 in one or more aspects. The garment 60 includes a crotch portion 64. The crotch portion 64 of the garment 60 may be similar or identical to the crotch portion 14, 44, or 54 in one or more aspects. The crotch portion 64 is positioned over the region of the urethra of the wearer (e.g., within the region of the crotch) when the garment 60 is worn. A fluid collection device (e.g., 100) may be secured to the crotch portion 64 with one or more securement features, such that fluids in the region (e.g., urine) may be collected by the fluid collection device. Accordingly, the garment 60 and crotch portion 64 may serve to align the fluid collection device with the urethra of the user such that fluids originating therefrom can be collected before soiling clothing or bedding.

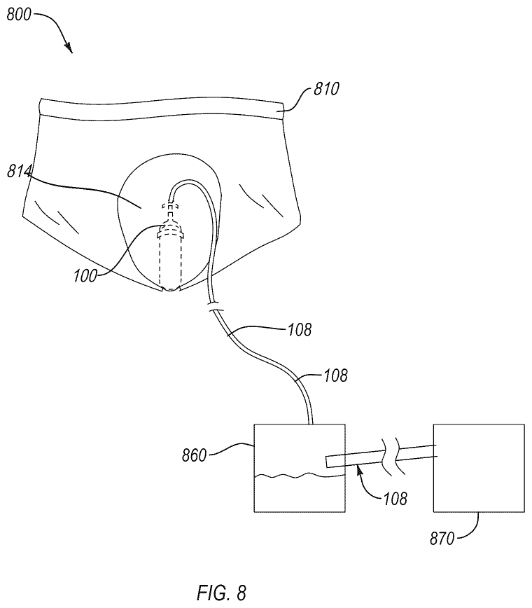

FIG. 7 is a front view of a garment 70 for fluid collection, according to an embodiment. The garment 70 may be similar or identical to the garment 10, 40, 50, or 60 in one or more aspects and, unless otherwise noted, may include any of the technical effects described above in relation to the garment 10. The garment 70 includes a fabric body 72 that is sized and shaped to fit on a pelvis of a wearer and maintain position thereon. The fabric body 72 may be sized, shaped, and configured to form undergarments in the form of a jock strap. The fabric body 72 of the garment 70 may be similar or identical to the fabric body 12 in one or more aspects. The garment 70 includes a crotch portion 74. The crotch portion 74 of the garment 70 may be similar or identical to the crotch portion 14, 44, 54, or 64 in one or more aspects. The crotch portion 74 is positioned over the region of the urethra of the wearer (e.g., within the region of the crotch) when the garment 70 is worn. A fluid collection device (e.g., 100) may be secured to the inner surface of the crotch portion 74 with one or more securement features, such that fluids in the region (e.g., urine) may be collected by the fluid collection device. Accordingly, the garment 70 and crotch portion 74 thereon may serve to align the fluid collection device with the urethra of the user such that fluids originating therefrom can be collected before soiling clothing or bedding.

In some examples, the garment may be adjustable to provide the technical effect of adjusting the position of the crotch portion 74 with respect to the urethral region of the wearer. For example, the garment may include one or more adjustable straps 78. The one or more adjustable straps 78 may attach to crotch portion 74 and the waistband 76. The waistband 76 and the one or more adjustable straps 78 may have complementary connections (e.g., button and button holes, hook and loop strips, etc.). The adjustable straps 78 may include multiple connection features (e.g., multiple buttons, hook and loop strips, etc.) along the body of the strap to allow adjustment of the length of the adjustable straps between the waistband 76 and the crotch portion 74. In some examples, the waistband 76 may be expandable or contractible. The adjustable straps 78 may provide the technical effect of selectively changing the location of the fluid collection device with respect to the urethral region of the wearer. Accordingly, the garment 70, adjustable straps 76, and crotch portion 74 may result in the technical effect of aligning the fluid collection device with the urethra of the user such that fluids originating therefrom can be collected before soiling clothing or bedding.

Any of the garments disclosed herein may be adjustable in one or more dimensions to selectively adjust the position of the crotch portion and fluid collection device therein with respect to a urethral region of the wearer. Accordingly, a single garment may be used for a wide variety of sizes of users.

FIG. 8 is a block diagram of a system 800 for fluid collection, according to an embodiment. The system 800 includes a garment 810 having a fluid collection device 100 secured thereto on a crotch portion 814, a fluid storage container 860, and a vacuum source 870. The fluid collection device 100, the fluid storage container 860, and the vacuum source 870 may be in fluid communication with each other via one or more conduits 108. For example, fluid collection device 100 may be operably coupled to (e.g., in fluid communication with) one or more of the fluid storage container 860 or the portable source 870 via the conduits 108. The garment 810 may be similar or identical to any of the garments disclosed herein, such as garment 10, 40, 50, 60, or 70. The fluid collection device 100 may be similar or identical to any of the fluid collection devices disclosed herein, such as a male or female fluid collection device.

Fluid (e.g., urine or other bodily fluids) collected in the fluid collection device 100 may be removed from the fluid collection device 100 via the conduit 108 which protrudes into an interior region of the fluid collection device 801. For example, a first open end of the conduit 108 may extend into the fluid collection device 100 to a reservoir therein. The second open end of the conduit 108 may extend into the fluid collection device 100 or the vacuum source 870. The suction force may be introduced into the interior region of the fluid collection device 100 via the first open end of the conduit 108 responsive to a suction (e.g., vacuum) force applied at the second end of the conduit 108. The suction force may be applied to the second open end of the conduit 108 by the vacuum source 870 either directly or indirectly.

The suction force may be applied indirectly via the fluid storage container 860. For example, the second open end of the conduit 108 may be disposed within the fluid storage container 860 and an additional conduit 108 may extend from the fluid storage container 860 to the vacuum source 870. Accordingly, the vacuum source 870 may apply suction to the fluid collection device 100 via the fluid storage container 860. In some examples, the suction force may be applied directly via the vacuum source 870. For example, the first open end of the conduit 108 may be disposed in the fluid collection device 100 and the second open end of the conduit 108 may be disposed within the vacuum source 870. An additional conduit 108 may extend from the vacuum source 870 to a point outside of the fluid collection device 100, such as to the fluid storage container 860. In such examples, the vacuum source 870 may be disposed between the fluid collection device 100 and the fluid storage container 860.

In examples, the fluid storage container 860 may include a bag (e.g., drainage bag), a bottle or cup (e.g., collection jar), tubing, or any other container for storing bodily fluids such as urine. In examples, the conduit 108 may extend from the fluid collection device 100 and attach to the fluid storage container 860 at a first point therein. An additional conduit 108 may attach to the fluid storage container 860 at a second point thereon and may extend and attach to the vacuum source 870. For example, the fluid storage container 860 may include a container in fluid communication with a first conduit section that is also in fluid communication with the fluid collection device 100. The container may be in fluid communication with a second section of the conduit 108 that is also in fluid communication with the vacuum source. In such examples, the vacuum source 870 may provide a vacuum/suction through the container to the fluid collection device 100 to provide suction in the chamber of the fluid collection device. Accordingly, a vacuum (e.g., suction) may be drawn through fluid collection device 801 via the fluid storage container 860. As the fluid is drained from the chamber, the fluid may travel through the first section of conduit to the fluid storage container where it may be retained. Fluid, such as urine, may be drained from the fluid collection device 100 using the vacuum source 870.

The vacuum source 870 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 870 may provide a vacuum or suction to remove fluid from the fluid collection device 100. In examples, the vacuum source 870 may be powered by one or more of a power cord (e.g., connected to a power socket), one or more batteries, or even manual power (e.g., a hand operated vacuum pump). The vacuum sources 870 disclosed herein may include one or more of a switch, a button, a plug, a remote, or any other device suitable to activate the vacuum source 870.

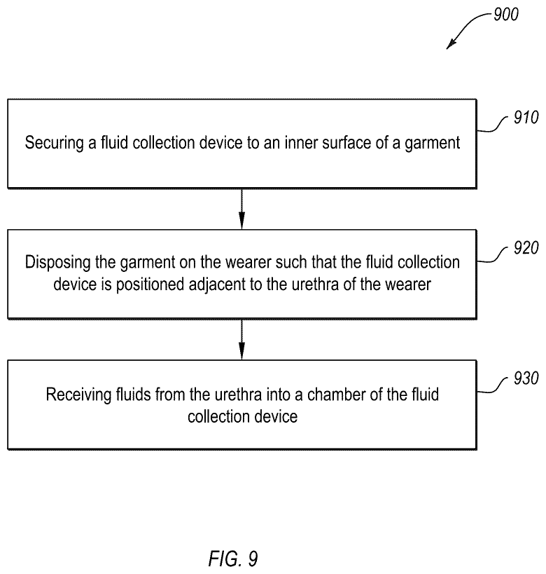

FIG. 9 is a flow diagram of a method 900 to collect fluid, according to an embodiment. The method 900 can include act 910, which recites “securing a fluid collection device to an inner surface of a garment.” Act 910 may be followed by act 920, which recites “disposing the garment on the wearer such that the fluid collection device is positioned adjacent to the urethra of the wearer.” Act 920 may be followed by act 930, which recites “receiving fluids from the urethra into a chamber of the fluid collection device.”

Acts 910, 920, 930 of the method 900 are for illustrative purposes. For example, the act 910, 920, 930 of the method 900 can be performed in different orders, split into multiple acts, modified, supplemented, or combined. In an example, one or more of the acts 910, 920, 930 of the method 900 can be omitted from the method 900. For example, the method 900 may not include the act 910. Any of the acts 910, 920, or 930 can include using any of the garments, fluid collection devices, vacuum sources, fluid storage containers, systems, or components of the same disclosed herein. For example, the garment may include a crotch portion having an inner surface with one or more securement features secured thereto. The fluid collection device may include a fluid impermeable barrier at least partially defining the chamber, the fluid impermeable barrier also defining an opening extending therethrough, the opening configured to be positioned adjacent to a female urethra or have a male urethra positioned therethrough. The fluid collection device may include a wicking material disposed with the chamber and a conduit disposed within wicking material, the conduit including an inlet positioned within the fluid collection device and an outlet.

Act 910 recites “securing a fluid collection device to an inner surface of a garment.” In some examples, act 910 includes at least one of (1) securing a first end region of a fluid impermeable barrier of the fluid collection device in a through hole defined by a portion of the inner surface of a crotch portion and a strap secured to the inner surface, (2) inserting a second end region of the fluid impermeable barrier of the fluid collection device in a pocket on the inner surface of the pocket, or (3) inserting a conduit to an additional through hole defined by a portion of the inner surface and an additional strap secured to the inner surface. Act 910 may include multiple acts, such as any combination of securing the first end region with the strap, inserting the second region in the pocket, and/or inserting the conduit in the through hole formed with the additional strap.

In some embodiments, one or more of the straps on the inner surface of the crotch portion may be at least partially detachably securable to the inner surface of the crotch portion. The act of securing a first end region of a fluid impermeable barrier of the fluid collection device in a through hole defined by a portion of the inner surface of a crotch portion and a strap secured to the inner surface, then, may include positioning the strap over the first end region of the fluid impermeable device with the fluid impermeable device between the strap and the inner surface, and then securing an end of the strap to the inner surface.

The fluid collection device may be a female fluid collection device and the garment may include a crotch portion configured to secure the female fluid collection device therein. The fluid collection device may be a male fluid collection device and the garment may include a crotch portion configured to secure the male fluid collection device therein. The crotch portion may be positioned on the garment according to the sex of the intended wearer.

Act 920 recites, “disposing the garment on the wearer such that the fluid collection device is positioned adjacent to the urethra of the wearer.” Disposing the garment on the wearer such that the fluid collection device is positioned adjacent to the urethra of the wearer can include donning the garment or placing the garment on the wearer prior to, after, or contemporaneously with positioning a fluid collection device on the inner surface of the crotch portion of a garment. Disposing the garment on the wearer such that the fluid collection device is positioned adjacent to the urethra of the wearer may include disposing the garment on the wearer such that the fluid collection device is positioned adjacent to the urethra of the wearer when the fluid collection device is positioned therein. For example, disposing the garment on the wearer such that the fluid collection device is positioned adjacent to the urethra of the wearer may be performed prior to act 910. Disposing the garment on the wearer such that the fluid collection device is positioned adjacent to the urethra of the wearer can include donning or placing any of the garments disclosed herein on the wearer. For example, disposing the garment on the wearer such that the fluid collection device is positioned adjacent to the urethra of the wearer may include adjusting one or more dimensions of the garment with adjustable straps thereon to place the crotch portion or the fluid collection device secured thereto over the urethra of the wearer.

In some examples, disposing the garment on the wearer such that the fluid collection device is positioned adjacent to the urethra of the wearer can include positioning the opening of a female fluid collection device such that the fluid permeable membrane of the female fluid collection device abuts or is positioned proximate to the female urethra. For example, positioning an opening of a fluid collection device adjacent to a female urethra of a user may include positioning opening of the fluid collection device, secured to the inner surface of the crotch portion of the garment, on or between the labia of a wearer.

Act 930 recites “receiving fluids from the urethra into a chamber of the fluid collection device.” For example, act 930 can include wicking the fluids away from urethra via the opening using wicking material (e.g., fluid permeable membrane and a fluid permeable support). In some examples, act 930 can include receiving the fluids into the chamber of the fluid collection device. In either example, act 930 can include flowing the fluid towards a portion of the chamber that is in fluid communication with an inlet of a conduit in fluid communication a vacuum source. For instance, act 930 can include flowing the fluids to a substantially unoccupied portion of the chamber (e.g., a reservoir), to a gravimetrically low point of the chamber, etc.

The method 900 may include applying suction with a vacuum source effective to suction the fluids from the chamber via a conduit disposed therein that is in fluid communication with the vacuum source. In examples, applying suction with a vacuum source effective to suction the fluids from the chamber via a conduit disposed therein that is in fluid communication with the vacuum source can include using any of the vacuum sources disclosed herein. In an example, applying suction can include activating the vacuum source (e.g., suction device) in fluid communication with the inlet of the conduit in the fluid collection device. In examples, activating the vacuum source in fluid communication with the inlet of the conduit in the fluid collection device can include supplying power to the vacuum source by one or more of flipping an on/off switch, pressing a button, plugging the vacuum source into a power outlet, putting batteries into the vacuum source, etc. In examples, the vacuum source may include a hand operated vacuum pump and applying suction with a vacuum source may include manually operating the hand operated vacuum pump effective to suction the fluids from the chamber via the conduit disposed therein that is in fluid communication with the vacuum source.

In examples, applying suction with a vacuum source effective to suction the fluids from the chamber via a conduit disposed therein that is in fluid communication with the vacuum source can be effective to remove at least some fluid (e.g., urine) from the chamber (e.g., interior region) of the fluid collection device. In examples, applying suction with a vacuum source effective to suction the fluids from the chamber via a conduit disposed therein that is in fluid communication with the vacuum source can be effective to transfer at least some of the fluid from the chamber of the fluid collection device to a fluid storage container (e.g., a bottle or bag) in fluid communication with the vacuum source and the fluid collection device. In examples, the vacuum source may be spaced from the fluid collection device.

In examples, applying suction with a vacuum source effective to suction the fluids from the chamber via a conduit disposed therein that is in fluid communication with the vacuum source may include detecting moisture in the chamber (e.g., via one or more moisture sensors) and responsive thereto, activating the vacuum source to provide suction in the chamber. Control of the vacuum source responsive to the signals indicating that moisture or a threshold level thereof is present in the chamber can be automatic, such as via a controller, or may merely provide an indication that a level of moisture is present that may necessitate removal of fluid from the chamber of the fluid collection device. In the latter case, a user may receive the indication and activate the vacuum pump.

In an example, the method 900 can include collecting the fluids that are removed from the fluid collection device, such as into a fluid storage container that is spaced from the fluid collection device that is in fluid communication with the conduit. The fluid storage container can include any of the fluid storage containers disclosed herein.

In an example, the fluid collection device (such as a soiled fluid collection device) may be removed from the garment and replaced with a replacement fluid collection device. Accordingly, the method 900 can include an act of removing the fluid collection device from the garment and an act of securing a replacement fluid collection device to the inner surface of the garment. Securing the replacement fluid collection device to the inner surface of the garment may include at least one of (1) securing a first end region of a fluid impermeable barrier of the replacement fluid collection device in the through hole defined by the portion of the inner surface of the crotch portion and the strap secured to the inner surface, (2) inserting a second end region of the fluid impermeable barrier of the replacement fluid collection device in the pocket on the inner surface of the pocket, or (3) inserting a conduit to the additional through hole defined by the portion of the inner surface and the additional strap secured to the inner surface.

Devices and methods described herein can be configured to collect urine from a male user, such as having a fluid collection device shaped and sized to receive a male urethra (e.g., penis) therein. In examples, the method 900 can include positioning a receptacle of a male fluid collection device around the male urethra such that the male urethra is positioned in the receptacle.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

1. A fluid collection system, comprising:
a garment configured to be worn on a pelvis of a user, the garment including at least a crotch portion having an inner surface, a first securement feature and a second securement feature spaced from the first securement feature; and
a fluid collection device having at least a portion configured to secure to at least one of the first securement feature or the second securement feature, the fluid collection device including:
a fluid impermeable barrier at least partially defining a chamber, an opening extending longitudinally along the fluid impermeable barrier and configured to be positioned adjacent to a urethra of the user, and an aperture configured to receive a conduit therethrough; and
a fluid permeable body positioned at least partially within the chamber to extend across at least a portion of the opening and configured to wick fluid away from the opening,
wherein the first securement feature and the second securement feature are positioned on the crotch portion of the garment and the fluid collection device is sized to position the opening and the fluid permeable body at least proximate to the urethra of the user.

2. The fluid collection system of claim 1, wherein the opening in the fluid impermeable barrier is positioned between the first securement feature and the second securement feature when the fluid collection device is secured to the garment.

3. The fluid collection system of claim 2, further comprising:
a conduit extending through the aperture and having a first portion disposed outside the chamber and a second portion disposed within the chamber; and
wherein the first securement feature is configured to secure a segment of the first portion of the conduit to the crotch portion.

4. The fluid collection system of claim 3, wherein the fluid impermeable barrier includes a second end region distal to the aperture and the second securement feature of the garment is configured to secure the second end region to the crotch portion with the opening in the fluid impermeable barrier disposed between the second securement feature and the first securement feature.

5. The fluid collection system of claim 2, wherein the fluid impermeable barrier includes a first end region between the opening and the aperture, and the first securement feature is positioned on the crotch portion of the garment to secure the first end region of the fluid impermeable barrier to the crotch portion of the garment.

6. The fluid collection system of claim 5, wherein the fluid impermeable barrier includes a second end region distal to the aperture and the second securement feature of the garment is configured to secure the second end region to the crotch portion with the opening in the fluid impermeable barrier disposed between the second securement feature and the first securement feature.

7. The fluid collection system of claim 6, further comprising:
a conduit extending through the aperture and having a first portion disposed outside the chamber and a second portion disposed within the chamber; and
an additional securement feature positioned on the crotch portion and configured to secure a segment of the first portion of the conduit to the crotch portion.

8. The fluid collection system of claim 1, wherein at least a portion of the first securement feature is releasably secured to the inner surface.

9. The fluid collection system of claim 1, wherein the crotch portion includes a wicking material.

10. The fluid collection system of claim 1, further comprising a conduit extending through the aperture and having a first portion disposed outside the chamber and a second portion disposed within the chamber, wherein the crotch portion includes an opening sized to receive the conduit therethrough.

11. The fluid collection system of claim 1, wherein the garment includes one or more adjustable straps configured to alter a position of the crotch portion and the fluid collection device with respect to the urethra of the user.

12. A fluid collection system, comprising:
a garment configured to be worn on a pelvis of a user, the garment including at least a crotch portion having an inner surface and a first securement feature secured to the inner surface of the garment;
a fluid collection device including:
a fluid impermeable barrier at least partially defining a chamber, an opening extending longitudinally along the fluid impermeable barrier and configured to be positioned adjacent to a urethra of the user, and an aperture configured to receive a conduit therethrough; and
a fluid permeable body positioned at least partially within the chamber to extend across at least a portion of the opening and configured to wick fluid away from the opening; and
a conduit extending through the aperture such that a first portion of the conduit is disposed outside the chamber and a second portion of the conduit is disposed within the chamber, the conduit having at least a portion sized and shaped to fit within the through hole,
wherein the first securement feature is positioned on the crotch portion of the garment to secure the second portion of the conduit to the garment and the fluid collection device is sized to position the opening and the fluid permeable body at least proximate to the urethra of the user.

13. The fluid collection system of claim 12, wherein at least a portion of the securement features is releasably secured to the inner surface.

14. The fluid collection system of claim 10, wherein the fluid impermeable barrier includes a second end region distal to the aperture and the garment includes a second securement feature on the inner surface of crotch portion spaced from the first securement feature configured to secure the second end region of the fluid impermeable barrier to the crotch portion.

15. The fluid collection system of claim 12, wherein the crotch portion includes a wicking material.

16. The fluid collection system of claim 12, wherein the garment includes a fluid absorption material.

17. The fluid collection system of claim 12, wherein the crotch portion includes an opening sized to receive the conduit therethrough.

18. The fluid collection system of claim 12, wherein the garment includes one or more adjustable straps configured to alter a position of the crotch portion and the fluid collection device with respect to the urethra of the user.

19. A method of collecting fluid, the method comprising:
providing a fluid collection device including a fluid impermeable barrier at least partially defining a chamber, an opening extending longitudinally along the fluid impermeable barrier and configured to be positioned adjacent to a urethra of the user, and an aperture at a first end region configured to receive a conduit therethrough, the fluid collection device also including a fluid permeable body positioned at least partially within the chamber to extend across at least a portion of the opening and configured to wick fluid away from the opening;
securing a first portion of the fluid collection device to an inner surface of a garment with a first securement feature on a crotch portion of the garment;
securing a second end region of the fluid collection device to the inner surface of the garment with a second securement feature on the crotch portion of the garment, the second end region being opposite to the first end region of the fluid impermeable barrier;
disposing the garment on the wearer such that the fluid permeable body disposed in the chamber is positioned adjacent to the urethra of the wearer through the opening in the fluid impermeable barrier; and
receiving fluids from the urethra into the chamber.

20. The method of claim 19, wherein the opening in the fluid impermeable barrier is positioned between the first securement feature and the second securement feature when the fluid collection device is secured to the garment.

21. The method of claim 20, wherein securing a first portion of the fluid collection device to an inner surface of a garment with a first securement feature on a crotch portion of the garment includes securing a conduit to the inner surface of the garment with the first securement feature, the conduit extending through the aperture and into the chamber.

22. The method of claim 21, further comprising securing the first end region of the fluid impermeable barrier to the crotch portion with an additional securement feature secured to the crotch portion between the first securement feature and the second securement feature.

23. The method of claim 20, wherein securing a first portion of the fluid collection device to an inner surface of a garment includes securing the first end region of the fluid impermeable barrier to the crotch portion with the first securement feature.

24. The method of claim 19, further comprising extending a conduit an opening in the crotch portion, the conduit having a first portion disposed outside the chamber and a second portion disposed within the chamber.

25. The method of claim 19, further comprising altering a position of the crotch portion of the garment and the fluid collection device with respect to the urethra of the user with one or more adjustable straps.

26. The method of claim 19, further comprising removing the fluid collection device from the garment and securing a replacement fluid collection device to the inner surface of the garment.
